(19) United States
(12) Patent Application Publication
(10) Pub. No.: US 2023/0301587 A1
(43) Pub. Date: Sep. 28, 2023

(54) SLEEP/WAKING DETECTION

(71) Applicant: Loewenstein Medical Technology S.A., Luxembourg (LU)

(72) Inventors: Farzad SAMIE, Karlsruhe (DE); Matthias Schwaibold, Karlsruhe (DE)

(21) Appl. No.: 18/186,280

(22) Filed: Mar. 20, 2023

(30) Foreign Application Priority Data
Mar. 22, 2022 (DE) 102022106673.8

(51) Int. Cl. A61B 5/00; A61B 5/0205; A61B 5/11

(52) U.S. Cl. CPC A61B 5/4812; A61B 5/0205; A61B 5/11; A61B 5/7264

(57) ABSTRACT

A method for determining at least one sleeping phase and/or one waking phase for at least one time span and a system having a control unit for determining at least one sleeping phase and/or one waking phase for at least one time span comprising a sensor for acquiring at least one movement signal, a sensor for acquiring at least one cardiac signal, a sensor for acquiring at least one respiration signal, a monitoring unit, which is configured to determine and/or assess a presence and/or a quality of the acquired movement, cardiac and respiration signals and one or more classification units, which are configured to determine a sleeping phase and/or waking phase on the basis of the determinations and assessments of the monitoring unit from the movement signals and/or the cardiac signals and/or the respiration signals.

### CROSS-REFERENCE TO RELATED APPLICATIONS

The present application claims priority under 35 U.S.C. § 119 of German Patent Application No. 102022106673.8, filed Mar. 22, 2022, the entire disclosure of which is expressly incorporated by reference herein.

### BACKGROUND OF THE INVENTION

### 1. Field of the Invention

The present invention relates to a system and a method for sleep/waking detection.

### 2. Discussion of Background Information

A large number of people suffer from poor sleep, which results in fading concentration and reduced intellectual performance. Poor sleep can also be responsible for disturbances of other functions such as muscle tension, respiration, heart rate, blood pressure, body temperature, hormones, and metabolism. The causes for poor sleep can be manifold. Poor sleep often results from a sleep disturbance of the patient. Poor sleep can also be present, however, although it was not possible to determine a sleep disturbance. To be able to make a statement about the sleep quality of a patient and to be able to remedy or treat possible sleep disturbances or illnesses accordingly, it is necessary to carry out detailed sleep diagnostics.

In view of the foregoing it would be advantageous to have available a system which supplies an improved, simple, and reliable statement about the sleep of a patient.

### SUMMARY OF THE INVENTION

The present invention provides a system having a control unit for determining at least one sleeping phase and/or one waking phase for at least one time span comprising at least one sensor configured for acquiring at least one movement signal, at least one sensor configured for acquiring at least one cardiac signal, at least one sensor configured for acquiring at least one respiration signal, at least one monitoring unit, which is configured to determine and/or assess a presence and/or quality of the acquired movement signals and cardiac signals and respiration signals, one or more classification units, which are configured to determine at least a sleeping phase and/or a waking phase on the basis of the determinations and assessments of the monitoring unit from the movement signals and/or the cardiac signals and/or the respiration signals.

In some embodiments, the system is characterized in that the monitoring unit is configured to receive movement signals and/or cardiac signals and/or respiration signals from the sensors.

In some embodiments, the system is characterized in that at least one threshold value is stored in the monitoring unit and that the quality of existing movement signals and/or cardiac signals and/or respiration signals is assessed on the basis of the at least one predefined threshold value.

In some embodiments, the system is characterized in that at least one threshold value is stored in each case for the movement signals and the cardiac signals and the respiration signals, wherein the quality of existing movement signals and/or cardiac signals and/or respiration signals is assessed on the basis of their respective threshold values.

In some embodiments, the system is characterized in that the monitoring unit is configured to select existing movement signals and/or cardiac signals and/or respiration signals which are above or below the respective threshold value.

In some embodiments, the system is characterized in that the monitoring unit is configured to pass on the selected movement signals and/or cardiac signals and/or respiration signals.

In some embodiments, the system is characterized in that the system comprises one or more extraction units which are configured to receive selected signals from the monitoring unit and to determine at least one movement feature from the selected movement signals and/or at least one cardiac feature from the selected cardiac signals and/or at least one respiration feature from the selected respiration signals.

In some embodiments, the system is characterized in that the determined movement features are selected from the group activity count, zero crossing count, median, standard deviation, acceleration minima, acceleration maxima.

In some embodiments, the system is characterized in that the determined cardiac features are selected from the group heart rate, heartbeat, heartbeat interval, heart rate variability, pulse rate, pulse wave, pulse wave amplitude, spectral power density, oxygen saturation (SpO₂ or SaO₂), plethysmograph, increase angle of the pulse waves, decrease angle of the pulse waves, ratios of increase to decrease angles of the pulse waves, durations of pulse wave increases, durations of pulse wave decreases, ratios of increase to decrease durations, pulse wave maxima, pulse wave minima, PTT (pulse transit time), CWF (continuous wave fluctuation), pulse amplitude.

In some embodiments, the system is characterized in that the determined respiration features are selected from the group respiration frequency, peak flow, variability, respiratory gas volume per breath, respiratory gas flow, contour of the respiratory gas flow, inspirational or expirational tidal volume, restoration, inspiration duration, expiration duration, leakage, ratio of inspiration to expiration duration, respiratory minute volume.

In some embodiments, the system is characterized in that the extraction unit is configured to pass on the extracted movement features and/or cardiac features and/or respiration features.

In some embodiments, the system is characterized in that the classification units are configured to receive signals from the monitoring unit and/or features from the extraction units.

In some embodiments, the system is characterized in that the classification units are configured to statistically evaluate the signals and/or the features on the basis of at least one statistical key figure.

In some embodiments, the system is characterized in that the statistical key figure is selected from the group minimum, maximum, mean value, median, standard deviation, variance, span, distribution, sum, difference.

In some embodiments, the system is characterized in that the classification units are configured to determine at least one sleeping phase and/or one waking phase from the movement signals and/or the cardiac signals and/or the respiration signals and/or from their statistical evaluations.

In some embodiments, the system is characterized in that the classification units are configured to determine at least one sleeping phase and/or at least one waking phase from the movement features and/or the cardiac features and/or the respiration features and/or their statistical evaluations.

In some embodiments, the system is characterized in that the classification unit comprises three classification subunits, wherein the control unit is configured to activate one of the three classification subunits in each case based on the results of the monitoring unit as a function of the presence and/or the quality of the signals, wherein the first classification subunit is activated if all signals are present in satisfactory quality, wherein the second classification subunit is activated if only the movement signals and the cardiac signals are present in satisfactory quality, wherein the third classification subunit is activated if only the movement signals are present in satisfactory quality.

In some embodiments, the system is characterized in that the first classification subunit is configured to receive movement features and cardiac features and respiration features and to determine sleeping phases and/or waking phases from the movement features and the cardiac features and the respiration features.

In some embodiments, the system is characterized in that the second classification subunit is configured to receive movement features and cardiac features and to determine sleeping phases and/or waking phases from the movement features and the cardiac features.

In some embodiments, the system is characterized in that the third classification subunit is configured to receive movement features and to determine sleeping phases and/or waking phases from the movement features.

In some embodiments, the system is characterized in that the time span is predeterminable, wherein the time span is at least 10 minutes, preferably at least one hour, preferably at least 4 hours, particularly preferably at least 6 hours, wherein the time span corresponds, for example, to the duration of a night's sleep.

In some embodiments, the system is characterized in that the time span is divided into one or more time periods, wherein the time periods are at least 10 seconds, preferably at least 20 seconds, particularly preferably 30 seconds long.

In some embodiments, the system characterized in that the system is configured and designed to assign a sleeping phase and/or a waking phase to at least one, preferably each time period on the basis of the movement signals and/or the cardiac signals and/or the respiration signals.

In some embodiments, the system is characterized in that the system is configured and designed to assign a sleeping phase and/or a waking phase to at least one, preferably each analyzed time period on the basis of the movement features and/or the cardiac features and/or the respiration features.

In some embodiments, the system is characterized in that at least one limiting value is stored in the classification units and that the assignment of the sleeping phases and/or waking phases to the time periods is determined on the basis of the at least one predefined limiting value.

In some embodiments, the system is characterized in that a sleeping phase is determined for a time period if the values are above the one or the multiple limiting values and that a waking phase is determined for a time period if the values are below the one or the multiple limiting values, or vice versa.

In some embodiments, the system is characterized in that the assignment of the sleeping phases and/or waking phases to the time periods is determined on the basis of multiple limiting values, wherein respective limiting values are stored in the classification units and retrievable for each feature.

In some embodiments, the system is characterized in that the classification units are configured to determine the sleeping phases and/or waking phases per time period from at least one feature, preferably from at least two or more different features.

In some embodiments, the system is characterized in that the analysis of the different features is weighted differently, for which purpose at least one weighting value is stored in the classification units and retrievable, wherein a respective weighting value is preferably stored for each feature.

In some embodiments, the system is characterized in that the system is dynamic, wherein the control unit activates the three classification subunits once and/or multiple times during a time span as a function of the presence and the quality of the signals.

In some embodiments, the system is characterized in that the system is configured to determine a sleep quality based on the number of the sleeping phases and/or the waking phases.

In some embodiments, the system is characterized in that the sensor is a photoplethysmography (PPG) sensor, which is configured to determine PPG signals, which supply items of information with respect to the cardiac activity.

In some embodiments, the system is characterized in that the sensor is an actigraphy sensor, which is configured to determine actigraphy signals that supply items of information with respect to a physical movement.

In some embodiments, the system is characterized in that the sensor is a flow sensor which is configured to determine flow signals that supply items of information with respect to the respiration activity.

In some embodiments, the system is characterized in that the monitoring unit, the extraction unit, and the classification unit are designed as one or more computing units, which are configured to record and/or store and/or evaluate and/or assess and/or select and/or pass on and/or process measured values, wherein data sets, such as threshold values and/or limiting values and/or rules such as the weighting values are stored in the computing units, on the basis of which the measured values and/or the extracted features are evaluated by the computing units.

The invention also provides a method for determining at least one sleeping phase and/or one waking phase for at least one time span.

In a first step, at least one signal of at least one movement parameter and/or at least one signal of at least one cardiac parameter and/or at least one signal of at least one respiration parameter is acquired.

In a further step, the presence of the acquired movement signals and cardiac signals and respiration signals is determined and/or the quality of the acquired movement signals and cardiac signals and respiration signals is assessed.

In a further step, at least one sleeping phase and/or one waking phase is determined from the movement signals and/or the cardiac signals and/or the respiration signals on the basis of the determinations and assessments of the monitoring unit.

In some embodiments, the method is characterized in that at least one movement feature is determined from the movement signals and/or at least one cardiac feature is determined from the cardiac signals and/or at least one respiration feature is determined from the respiration signals.

In some embodiments, the method is characterized in that at least one sleeping phase and/or one waking phase is determined from the movement features and/or the cardiac features and/or the respiration features.

### BRIEF DESCRIPTION OF THE DRAWINGS

Exemplary embodiments of the system of the invention are shown in the drawings. In the drawings:

FIG. 1 shows an exemplary device, in which the system according to the invention can be at least partially integrated;

FIG. 2 shows shows exemplary determined signal curves, wherein the signals of a flow, an actigraphy, and a (photo)plethysmograph are represented graphically over time t;

FIG. 3 shows shows an exemplary schematic structure of a simple exemplary embodiment of the system according to the invention, which comprises three sensors, a monitoring unit, and a classification unit;

FIG. 4 shows shows an exemplary schematic structure of a further exemplary embodiment of the system according to the invention, wherein it additionally comprises an extraction unit, which is connected between the monitoring unit and the classification unit;

FIG. 5 shows shows an exemplary determined (photo)plethysmograph having an exemplary representation of extracted cardiac features; and

FIG. 6 shows shows an exemplary representation of an extracted cardiac feature in a coordinate system, wherein the heartbeats are plotted on the X axis and the heartbeat interval in seconds is plotted on the Y axis.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

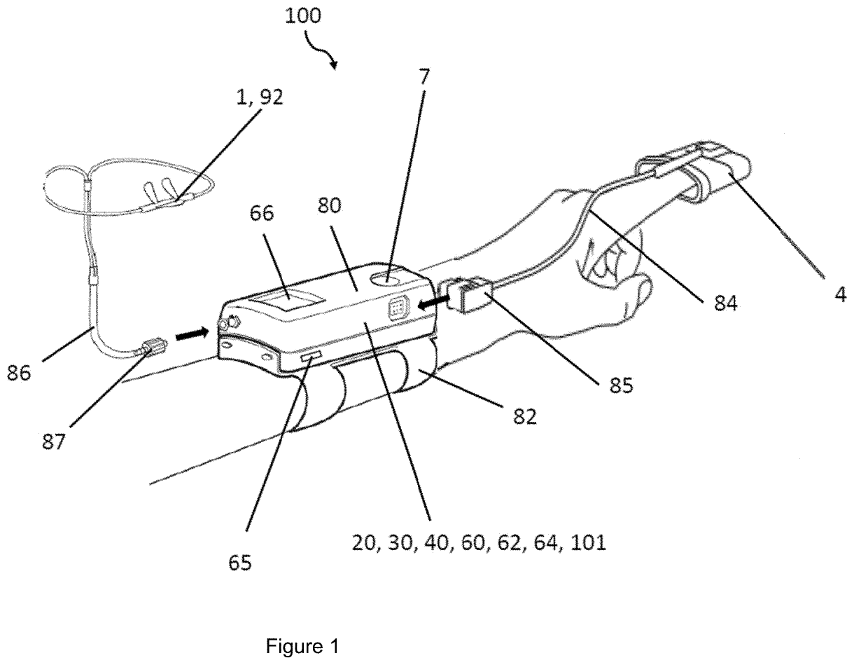

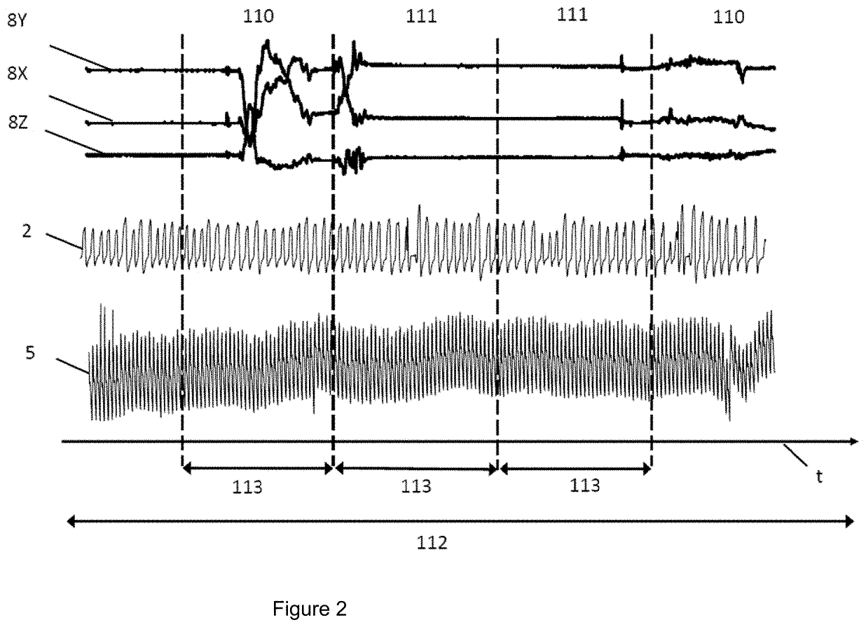

FIG. 1 shows an exemplary device 80, in which the system 100 according to the invention can be at least partially integrated. System 100 is configured and designed to determine at least one sleeping phase 110 and/or one waking phase 111 for at least one time span 112 (see FIG. 2).

System 100 can, for example, be at least partially integrated in one or more devices 80, which can be fastened, for example, on the body of a patient or user. Patient and user are used as synonyms herein and designate any individual who uses the system 100 according to the invention for determining sleeping phases 110 and/or waking phases 111 or on which it is applied. The device 80 can in some exemplary embodiments also not be fastened on the body and can be designed, for example, as a bedside device 80. The device 80 can receive data in a wired and/or wireless manner. It is thus also conceivable that the device 80 is located independently of the location of the patient and receives data wirelessly (not shown).

Device 80 is preferably fastened on the body of the patient. The fastening of the device 80 on the body of the patient can take place, for example, via one or more device belts 82. The device belt 82 is manufactured, for example, from a skin-friendly plastic. Another fastening on the body of the patient is also possible, for example via adhesive bonding on the skin or the like. The device 80 can be fastened, for example, on the wrist of a patient. Such a wrist device 80 is particularly pleasant to wear for the patient and does not significantly disturb the sleep.

System 100 comprises at least three sensors 1, 4, 7 for noninvasive measurement of at least one signal 2, 5, 8. The at least three sensors 1, 4, 7 are configured and designed to acquire signals 2, 5, 8 from at least three different categories. The categories relate at least to the physical movement and the cardiac activity and the respiration activity of the patient.

System 100 comprises at least one sensor 7 configured for acquiring at least one movement signal 8. Sensor 7 is configured and designed to determine movement signals 8 noninvasively. The sensor 7 can be, for example, an actigraphy sensor 7. The movement signal 8 can be, for example, an actigraphy signal 8. In this specific exemplary embodiment, the actigraphy sensor 7 can be configured to determine actigraphy signals 8, which supply items of information with respect to a physical movement.

The movement signal 8 can be determined in the exemplary embodiment according to FIG. 1 via a sensor 7 on the wrist. It is also conceivable that the sensor 7 is attached at another point of the body. Multiple sensors 7 at different points of the body of the patient are also conceivable to determine more differentiated statements about the physical movement.

Sensor 7 can be integrated, for example, in a device 80, which is worn on the wrist, for example. In an alternative embodiment, it is conceivable that the movement signal 8 is alternatively or additionally measured on the torso of the patient. For this purpose, the sensor 7 can be arranged in a device 80 which is fastened via a device belt 82 on the abdomen and/or on the thorax. Alternatively or additionally, sensor 7 can also be fastened, for example, on the lower extremities and/or on the head (not shown).

System 100 can in some embodiments comprise multiple sensors 7, which can acquire the physical movement at different points of the body of the patient. The acquired physical movements can be selected from the group of arm movements, wrist movements, hand movements, leg movements, foot movements, ankle movements, thorax movements, abdomen movements, head movements, eye movements.

The actigraphy sensor 7 can acquire one or more actigraphy signals 8. The actigraphy signals 8 can be determined in device 80. The actigraphy signals 8 can also be determined outside device 80 and transmitted, for example, into the device 80. The transmission of the actigraphy signals 8 takes place to computing units 20, 30, 40 for further processing of the actigraphy signals 8. These computing units 20, 30, 40 can be arranged, for example, in the device 80.

The actigraphy signals 8 can be recorded, for example, as actigraphy curves over time t. In one exemplary embodiment, for example, three actigraphy curves 8X, 8Y, 8Z can be recorded over time t (see FIG. 2). The three actigraphy curves in this exemplary embodiment describe the acceleration to which the actigraphy sensor 7 is subjected in the three movement directions X, Y, Z. In this case, the X direction could represent, for example, a movement to the left or right, the Y direction a movement forward or back, and the Z direction a movement upward or downward.

The system moreover comprises at least one sensor 4 configured for acquiring at least one cardiac signal 5. Sensor 4 is configured and designed to determine cardiac signals 5 noninvasively. A plethysmograph, preferably a photoplethysmograph is preferably determined and recorded using a pulse oximeter and/or a pulse spectrometer. The sensor 4 can be, for example, a photoplethysmography sensor, abbreviated as a PPG sensor 4. The cardiac signal 5 can be, for example, a photoplethysmography signal, abbreviated as a PPG signal 5. In this specific exemplary embodiment, the PPG sensor 4 can be configured to determine PPG signals 5, which supply items of information with respect to the cardiac activity.

The cardiac signal 5 can be measured in this exemplary embodiment according to FIG. 1 on the finger. It is also possible that the cardiac signal 5 is measured on the head, for example on the earlobe. A measurement on the foot, on the toe, on the torso, on the wrist, or on another suitable point of the body (not shown) is also conceivable.

The PPG sensor 4 can be integrated in the device 80. In the specific exemplary embodiment according to FIG. 1, the PPG sensor 4 is not integrated in the device 80. For example, the PPG sensor 4 is located in a pulse oximeter and/or a pulse spectrometer, which is fastened on a finger. Corresponding pulse oximeters and pulse spectrometers are known. The sensor 4 can acquire, for example, pulse-oximetry signals 5, such as an oxygen saturation (SpO₂) of the blood and/or a pulse rate of the patient. The pulse oximetry sensor 4 can essentially comprise at least one, preferably at least two light-emitting diodes and a receiver diode. The pulse-oximetry signals 5 can be acquired via the measurement of the light absorption or the light remission upon illumination through the skin.

For example, the PPG signal 5 can be determined on the finger. The PPG signal 5 can then be transmitted via a wired connection 84. The wired connection 84 can be connected via a fitting 85 to the device 80. A wireless transmission of the PPG signals 5, for example via Bluetooth, is also conceivable. For example, the PPG signal 5 is transmitted into the device 80, in which computing units 20, 30, 40 can be arranged for further processing of the PPG signal 5.

System 100 moreover comprises at least one sensor 1 configured for acquiring at least one respiration signal 2. Sensor 1 is configured and designed to determine respiration signals 2 noninvasively. The respiration signals 5 are selected from the group: flow (respiratory gas flow), volume (respiratory gas volume), pressure.

Sensor 1 can be, for example, a flow sensor 1. The respiration signal 2 can be, for example, a flow signal 2. In this specific exemplary embodiment, the flow sensor 1 can be configured to determine flow signals 2, which supply items of information with respect to the respiratory activity.

Signal 2 can be determined via a patient interface 92. Signal 2 is determined in the specific exemplary embodiment according to FIG. 1 via a patient interface 92, which is designed as a dynamic pressure nasal cannula 92 (illustration not to scale). The dynamic pressure nasal cannula 92 can acquire, in conjunction with a sensor 1 integrated in the device 80, for example, the flow and/or the pressure and/or the volume. Signal 2 can be transmitted via a hose connection 86. The hose connection 86 can be connected via a fitting 87 to the device 80. The fitting 87 can be designed, for example, as a Luer lock fitting. For example, the flow signal 2 is transmitted into device 80, in which computing units 20, 30, 40 can be arranged for further processing of the flow signal 2.

The movement signals 8 and/or cardiac signals 5 and/or respiration signals 2 can be recorded and represented, for example, on the basis of signal curves over time t. For this purpose, system 100 can comprise a display unit 66. The display unit 66 can comprise a monitor. The monitor can be integrated, for example, in device 80. In one preferred embodiment, the monitor can be configured and designed independently of device 80 and can receive and display the data from system 100.

In addition to the at least three sensors 1, 4, 7, the system 100 comprises at least one monitoring unit 20 and at least one classification unit 40. The monitoring unit 20 is configured to determine and/or assess a presence and/or a quality of the acquired movement signals 8 and cardiac signals 5 and respiration signals 2. The one or more classification units 40 are configured, on the basis of the determinations and assessments of the monitoring unit 20, to determine at least one sleeping phase 110 and/or one waking phase 111 from the movement signals 8 and/or the cardiac signals 5 and/or the respiration signals 2. In addition to the monitoring unit 20 and the classification unit 40, the system 100 can moreover comprise at least one extraction unit 30. The monitoring unit 20, the extraction unit 30, and the classification unit 40 are designed as one or more computing units, which are configured to record and/or store and/or evaluate and/or assess and/or select and/or pass on and/or process measured values 2, 5, 8.

In the specific exemplary embodiment according to FIG. 1, the computing units, namely the monitoring unit 20, the extraction unit 30, and the classification unit 40, can be stored in the device 80. External storage, for example in external computer systems, servers, or clouds is also conceivable.

The system 100 moreover comprises a control unit 101. The control unit 101 is configured and designed to control the different computing units 20, 30, 40 of the system 100. The control unit 101 can also be stored in the device 80 or externally.

The system 100 can comprise at least one memory unit 60. The memory unit 60 can also be stored in the device 80 or externally. The memory unit 60 can be configured to temporarily store at least one signal and make it available for further processing. The memory unit 60 can be configured to store acquired and/or determined data for later readout. In addition, predefined data sets such as threshold values 22 and/or limiting values 42 and/or rules can be stored in the memory unit 60, on the basis of which measured values or signals 2, 5, 8 and/or features 3, 6, 9 extracted from the signals 2, 5, 8 can be evaluated by the computing units 20, 30, 40.

The system 100 can comprise an energy supply 62. The energy supply 62 can take place via batteries and/or accumulators. Alternatively or additionally, the energy supply 62 can take place externally via power cables.

The system 100 can comprise a data transmission unit 64. The input and/or output of data can take place via the data transmission unit 64. The data transmission can take place in a wired and/or wireless manner. The data transmission can take place via an interface 65. The interface 65 can be configured as wired, as an infrared interface, as a Bluetooth interface, or as a USB interface. For example, mobile wireless or near field data and/or WLAN and/or Bluetooth and/or network data can be received or transmitted via the interface 65.

FIG. 2 shows exemplary determined signal curves, wherein the signals of a flow 2, an actigraphy 8, and a (photo)plethysmograph 5 are shown graphically over time t.

The determined signals 2, 5, 8 can be graphically represented by means of signal curves over time t. The signal curves can be output and displayed, for example, via the display unit 66.

System 100 is configured and designed so that a time span 112 is predeterminable. The patient or medical personnel can predetermine the time span 112. The start and the end of the time span 112 can be preset. The time span 112 is at least 10 minutes. For example, the time span 112 is at least one hour, preferably at least 2 hours, particularly preferably at least 6 hours. In one advantageous embodiment, the time span 112 corresponds, for example, to the duration of a night's sleep. FIG. 2 shows an exemplary time span 112, which does not correspond to a night's sleep. A detail of a time span 112 is shown in FIG. 2.

The time span 112 can be divided into one or more time periods 113. The time periods 113 are at least 10 seconds, preferably at least 20 seconds long. In a specific exemplary embodiment, the time period 113 is 30 seconds long. This means that the time periods 113 each comprise, for example, 30 seconds.

System 100 is configured and designed to assign a sleeping phase 110 and/or a waking phase 111 to at least one time period 113 on the basis of the movement signals 8 and/or the cardiac signals 5 and/or the respiration signals 2. Preferably, the system 100 assigns a sleeping phase 110 and/or a waking phase 111 to each time period 113 of the time span 112. Due to the selection of the three signal categories 8, 5, 2, sufficient signals 2, 5, 8 are always available according to the invention to preferably assign a sleeping phase 110 and/or a waking phase 111 to each time period 113.

FIG. 2 shows by way of example that a time period 113 which the system 100 assigns a sleeping phase 110 is followed by two time periods 113 which the system 100 each assigns a waking phase 111. The two waking phases 111 are in turn followed by a time period 113 which the system 100 assigns a sleeping phase 110.

In this way, the system 100 can preferably assign all time periods 113 of the entire time span 112 sleeping phases 110 and/or waking phases 111. A particularly reliable system 100 for determining sleeping phases 110 and/or waking phases 111 is provided by the system 100 according to the invention having the at least three sensors 1, 4, 7.

The goal of the invention is primarily not to diagnose a specific sleep-related disease or disturbance. The goal of the invention is rather to ensure a detailed and reliable determination of the sleeping phases 110 and waking phases 111 within a time span 112. The time span 112 preferably comprises at least one night's sleep of a patient here. A particularly reliable determination of the sleeping phases 110 and waking phases 111 is provided by the selection from at least three signals, namely at least one movement signal 8, at least one cardiac signal 5, and at least one respiration signal 2.

Conversely, however, it is possible to also draw conclusions about specific sleep-related diseases or disturbances or about a sleep quality 120 due to the detailed and reliable determination of the sleeping phases 110 and waking phases 111 within the time span 112.

The system 100 is thus also configured in particular to determine a sleep quality 120 based on the number of the sleeping phases 110 and/or the waking phases 111. The sleep quality 120 can make a statement about whether the sleep of one or more preceding time spans 112 was overall “good sleep” or “bad sleep”.

The sleep quality 120 can be output, for example, as a sleep quality index. The sleep quality index can be output, for example, in the form of a number, a symbol, a graphic, or the like and can thus be user-friendly and easy to read out. Users without specialized medical knowledge can therefore also receive a statement about the sleep quality 120 by way of the system 100, wherein a low sleep quality index indicates worse sleep and a higher sleep quality index indicates better sleep. It can therefore be inferred easily from the system 100 how the sleep quality 120 of one or more preceding time spans 112 was.

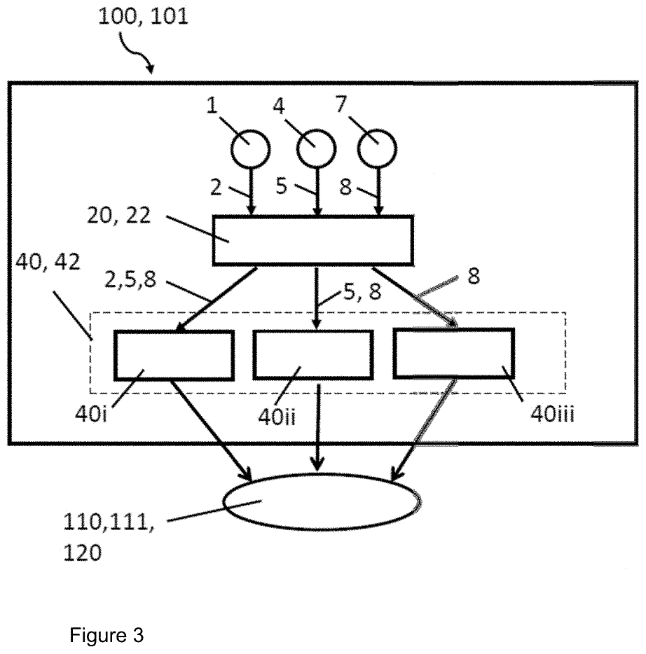

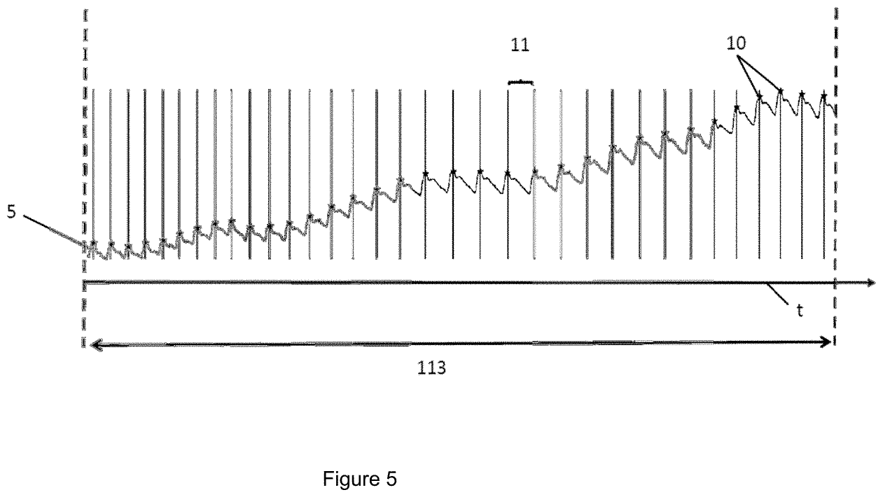

FIG. 3 shows an exemplary schematic structure of a simple exemplary embodiment of the system 100 according to the invention, which comprises three sensors 1, 4, 7, a monitoring unit 20, and a classification unit 40. FIG. 4 shows an exemplary schematic structure of a further exemplary embodiment of the system 100 according to the invention, wherein it additionally comprises an extraction unit 30 that is connected between the monitoring unit 20 and the classification unit 40.

In the specific exemplary embodiments according to FIG. 3 and FIG. 4, the three sensors, namely, for example, the flow sensor 1, the PPG sensor 4, and the actigraphy sensor 7, respectively acquire flow signals 2, PPG signals 5, and actigraphy signals 8. The acquired signals 2, 5, 8 are passed on to the monitoring unit 20. The acquired signals 2, 5, 8 can be passed on in either a wired and/or wireless manner.

The monitoring unit 20 is configured to receive movement signals 8 and/or cardiac signals 5 and/or respiration signals 2 from the sensors 1, 4, 7. The monitoring unit 20 then determines which signals 2, 5, 8 are present. Moreover, the monitoring unit 20 assesses the quality of the acquired signals 2, 5, 8.

It has been shown that not all signals 2, 5, 8 are always to be acquired or measured in a time span 112 to be measured. Moreover, it has been shown that not all signals 2, 5, 8 are always present in a satisfactory quality. This can be because, for example, the patient moves during the time span 112 to be measured, due to which the sensors 1, 4, 7 can experience an unfavorable position change or transmission pathways of the signals 1, 4, 7 can be interrupted.

It has been shown that the movement signal 8 is in general always present in a satisfactory quality upon proper use.

The PPG signal 5 is usually present in a satisfactory quality. However, the PPG signal 5 can also be absent during the time span 112 to be measured, for example if the PPG sensor 4 slips or even slips off entirely from the finger. Unfavorable slipping of the PPG sensor 4 can be reversed again in principle by renewed patient movement and the absence of the PPG signal 5 could only affect a certain part of the time span 112 and thus be reversible. However, if the PPG sensor 4 were to slip off unnoticed and thus be permanently removed, the absence of the PPG signal 5 would be permanent and non-reversible.

The flow signal 2 is not always determinable or is not always to be acquired in satisfactory quality during a time span 112. With the respiration signal 2, patient movements can have the strongest negative effect on the measurement accuracy. For example, the patient interface 92 can slip unnoticed in sleep and/or the hose connection 86 can be interrupted. For example, the hose connection 86 can no longer be capable of transporting the flow due to kinking or pinching. These interruptions can generally be reversible and can occur more frequently during the time span 112.

To determine the presence of the signals 2, 5, 8 and/or to assess the quality of the acquired signals 2, 5, 8, at least one threshold value 22 can be stored in the monitoring unit 20. The quality of the movement signals 8 and/or cardiac signals 5 and/or respiration signals 2 can then be assessed on the basis of the at least one predefined threshold value 22. At least one predefined threshold value 22 is preferably stored in each case for each signal 2, 5, 8.

At least one threshold value 22 is preferably stored in each case for each signal category, thus for the movement signals 8 and the cardiac signals 5 and the respiration signals 2. The quality of existing movement signals 8 and/or cardiac signals 5 and/or respiration signals 2 can thus be assessed in each case on the basis of the corresponding threshold value 22. The assessment of the signals 2, 5, 8 takes place either on the basis of exceeding or on the basis of falling below the corresponding threshold value 22. A selection of the signals can take place on the basis of the assessment of the signals 2, 5, 8 on the basis of exceeding or falling below the corresponding threshold value 22. The selection can take place via the monitoring unit 20.

The monitoring unit 20 can be configured to select present movement signals 8 and/or cardiac signals 5 and/or respiration signals 2, which are above or below the respective threshold value 22. The monitoring unit 20 can furthermore be configured to pass on the selected movement signals 8 and/or cardiac signals 5 and/or respiration signals 2.

In one simple exemplary embodiment, the selected movement signals 8 and/or cardiac signals and/or respiration signals 2 can be passed on directly to the classification unit 40 (see FIG. 3).

In one preferred exemplary embodiment, the selected movement signals 8 and/or cardiac signals 5 and/or respiration signals 2 can also be passed on to the extraction unit 30 (see FIG. 4).

It is apparent from FIG. 3 that the system 100 comprises one or more classification units 40, which can be configured in a simple exemplary embodiment to determine at least one sleeping phase 110 and/or one waking phase 111 from the movement signals 8 and/or the cardiac signals 5 and/or the respiration signals 2. In one advantageous embodiment, the classification unit 40 can be configured to determine at least one sleeping phase 110 and/or one waking phase 111 from statistical evaluations of the movement signals 8 and/or the cardiac signals 5 and/or the respiration signals 2.

The system 100 can comprise a first classification subunit 40i, which can be configured to receive movement signals 8 and cardiac signals 5 and respiration signals 2 from the monitoring unit 20 and to determine sleeping phases 110 and/or waking phases 111 directly from the movement signals 8 and the cardiac signals 5 and the respiration signals 2. The system 100 can additionally comprise a second classification subunit 40ii, which can be configured to receive movement signals 8 and cardiac signals 5 from the monitoring unit 20 and to determine sleeping phases 110 and/or waking phases 111 from the movement signals 8 and the cardiac signals 5.

System 100 can furthermore comprise a third classification subunit 40iii, which can be configured to only receive movement signals 8 from the monitoring unit 20 and to determine sleeping phases 110 and/or waking phases 111 from the movement signals 8.

The classification unit 40 can thus comprise at least three subunits 40i, 40ii, 40iii, so that it is always ensured that one sleeping phase 110 or one waking phase 111 per time period 113 can be determined in each case from the available signals 2, 5, 8. In some embodiments, more than three subunits of the classification unit 40 can also be provided, which can process further possible present signal combinations.

The subunits 40i, 40ii, 40iii can be activated by the monitoring unit 20. The control can also be carried out by the classification unit 40 itself. In one exemplary embodiment, it is provided that the higher-order control unit 101 registers which combination of signals 2, 5, 8 it is and thereupon activates the correct subunit 40i, 40ii, or 40iii. Activate in this meaning means that the respective subunit 40i, 40ii, or 40iii receives the corresponding signals 2 and/or 5 and/or 8 for further processing.

The system 100 can thus be designed as dynamic, wherein the control unit 101 activates the classification subunits 40i, 40ii, 40iii once and/or multiple times during a time span 112 in dependence on the presence and the quality of the signals 2, 5, 8. The activation and thus the selection of the appropriate subunit 40i, 40ii, 40iii preferably takes place from the beginning for each individual time period 113.

FIG. 4 shows a preferred exemplary embodiment having at least one extraction unit 30. In this exemplary embodiment, the selected movement signals 8 and/or cardiac signals 5 and/or respiration signals 2 can be passed on to the extraction unit 30 (see FIG. 4). The extraction unit 30 is configured to receive the selected signals 2, 5, 8. The extraction unit 30 is configured to determine at least one movement feature 9 from the selected movement signals 8 and/or at least one cardiac feature 6 from the selected cardiac signals 5 and/or at least one respiration feature 3 from the selected respiration signals 2. The extraction unit 30 is furthermore configured to pass on the extracted movement features 9 and/or cardiac features 6 and/or the respiration features 3.

The extraction unit 30 can be subdivided in some embodiments into at least three subunits 30i, ii, and 30iii. The extraction subunits 30i, 30ii, 30iii can each be configured to receive a specific combination of signals 2, 5, 8 from the monitoring unit 20. The first extraction subunit 30i can be configured to receive selected movement signals 8 and cardiac signals 5 and respiration signals 2. The first extraction unit 30i is then activated when all three signals 2, 5, 8 are present in satisfactory quality. The second extraction subunit 30ii can be configured to receive selected movement signals 8 and cardiac signals 5. The second extraction unit 30ii is then activated if only two signals are present in satisfactory quality, namely the movement signals 8 and the cardiac signals 5. The third extraction subunit 30iii can be configured to receive selected movement signals 8. The third extraction unit 30iii is then activated when only the movement signal 8 is present in satisfactory quality. In some embodiments, more than three subunits of the extraction unit 30 can also be provided, which can process further possible provided signal combinations.

From the set of the signal data 2, 5, 8, the extraction unit 30 and/or its subunits 30i, 30ii, 30iii, which are designed as computing units, can recognize patterns, repetitions, and/or laws with the aid of pattern recognition and extract corresponding mathematical features 3, 6, 9.

The extraction unit 30 and/or its subunits 30i, 30ii, 30iii are therefore configured to determine and/or extract at least one feature 3, 5, 8 from the signals 2, 5, 8.

The extraction unit 30 can be configured to determine at least one movement feature 9 at least from the selected movement signals 8. The extraction unit 30 can be configured to determine at least one cardiac feature 6 at least from the selected cardiac signals 5. The extraction unit can be configured to determine at least one respiration feature 3 at least from the selected respiration signals 2.

At least one algorithm can be stored in the extraction unit 30, by means of which at least one feature 3, 6, 9 can be computed from one or more signals 2, 5, 8.

In the specific exemplary embodiment according to FIG. 4, the first extraction subunit 30i is configured to determine at least one movement feature 9 from the selected movement signals 8 and at least one cardiac feature 6 from the selected cardiac signals 5 and at least one respiration feature 3 from the selected respiration signals 2. The first extraction subunit 30i can accordingly extract at least one feature 3, 6, 9 from each of three signals 2, 5, 8 of different categories (physical movement; cardiac activity; respiration activity). The first extraction subunit 30i can also extract more than one feature 3, 6, 9 in each case.

In the specific exemplary embodiment according to FIG. 4, the second extraction subunit ii is configured to determine at least one movement feature 9 from the selected movement signals 8 and to determine at least one cardiac feature 6 from the selected cardiac signals 5. The second extraction subunit 30ii can accordingly extract at least one feature 6, 9 from each of two signals 5, 8 of different categories (physical movement; cardiac activity). The second extraction subunit 30ii can also extract more than one feature 3, 6 in each case.

In the specific exemplary embodiment according to FIG. 4, the third extraction subunit 30iii is configured to determine at least one movement feature 9 from the selected movement signals 8. The third extraction subunit 30iii can accordingly extract at least one feature 9 from a signal 8 (physical movement). The third extraction subunit 30iii can also extract more than one feature 9 from the signals 8.

The subunits 30i, 30ii, 30iii can be activated by the monitoring unit 20. The control can also be carried out by the extraction unit 30 itself. In one exemplary embodiment, it is provided that the higher-order control unit 101 registers which combination of signals 2, 5, 8 it is and thereupon activates the correct subunit 30i, 30ii, or 30iii. In each case, the respective subunit i, 30ii, or 30iii receives the corresponding signals 2 and/or 5 and/or 8 for further processing.

The different extraction units 30i, 30ii, 30iii can be activated once and/or multiple times during a time span 112, namely in dependence on the presence and quality of the signals 2, 5, 8—determined by the monitoring unit 20. The activation and thus the selection of the corresponding subunit 30i, 30ii, 30iii preferably takes place from the beginning for each individual time period 113.

The system 100 can be operated in conjunction with a ventilator 90 (not shown). The ventilator 90 can be configured to assist a patient or another user in the natural respiration and/or to take over the ventilation of a user or patient and/or can be used for respiratory therapy and/or can act on the respiration of a user or patient in another manner. Ventilator 90 can be configured for clinical or home applications. The ventilator 90 can be configured and designed in particular to carry out CPAP and/or APAP therapy.

The ventilator 90 has an interface for coupling a hose system 94, via which a respiratory airflow for ventilation or respiratory assistance can be supplied to the patient or user. For this purpose, a patient interface 92 can be connected to the hose system 94. A patient interface 92 is to be understood as any peripheral device which is designed for interaction with a living being. In particular, the patient interface 92 is designed for treatment and/or diagnostic purposes in conjunction with the ventilator 90. The patient interface 92 can be designed as a breathing mask, for example as a nasal cannula or oxygen cannula, nose mask, nose cushion mask, full face or total face mask, and tracheal tubes or cannulas. Sensor 1 for measuring the respiration signal 2 can be arranged in or on or in conjunction with the patient interface 92.

Examples are described hereinafter for
respiration features 3 extracted from the respiration signals 2
cardiac features 6 extracted from the cardiac signals 5
movement features 9 extracted from the movement signals 8.

The invention is not restricted to the described features 3, 6, 9.

Respiration features 3 can be derived from the respiration signals 2 (see FIG. 2) selected from the group: respiratory rate, peak flow, variability, respiratory gas volume per breath, respiratory gas flow, contour of the respiratory gas flow, inspirational or expirational tidal volume, restoration, inspiration duration, expiration duration, leakage, ratio of inspiration to expiration duration, respiratory minute volume.

Movement feature 9 can be derived from the movement signals 8 (see FIG. 2) selected from the group: movement count (activity count), zero crossing count, median, standard deviation, acceleration minima, acceleration maxima.

Cardiac features 6 can be derived from the PPG signals 5 (see FIG. 2) from the group: heart rate, heartbeat, heartbeat interval, heart rate variability, pulse rate, pulse wave, pulse wave amplitude, spectral power density, oxygen saturation (SpO₂ or SaO₂), plethysmograph, increase angle of the pulse waves, decrease angle of the pulse waves, ratios of increase to decrease angles of the pulse waves, durations of pulse wave increases, durations of pulse wave decreases, ratios of increase to decrease durations, pulse wave maxima, pulse wave minima, PTT (pulse transit time), CWF (continuous wave fluctuation), pulse amplitude.

FIG. 5 shows an exemplary determined (photo)plethysmograph over time t with an exemplary representation of extracted cardiac features 6.

The PPG signals 5 can be represented in a plethysmograph. The course of the PPG signal 5 is shown over time t in an exemplary time period 113 by way of example in FIG. 5. For example, one or more cardiac features 6 can be extracted on the basis of the plethysmograph. At least one algorithm can be stored for this purpose in the extraction unit 30, by means of which at least one cardiac feature 6 can be computed from one or more cardiac signals 5. The algorithm can be configured to carry out a feature detection. The PPG signals 5 have a periodic pattern. The algorithm can be configured to compute or derive at least one cardiac feature 6 from the periodic pattern. It is also possible to derive multiple cardiac features 6 on the basis of the plethysmograph.

For example, a feature detection is carried out from the PPG signals 5 on the basis of a detection of peaks (high points) 10 in the PPG profile (see FIG. 5). An algorithm for a detection of the peaks 10 can be stored for this purpose. The peaks 10 represent the heartbeat of the patient. Since these peaks 10 correspond to the heartbeat 10, among other things, the pulse wave and/or the heart rate 11 and/or the heart rate variability and the like can be derived from the peaks 10.

For example, the heart rate 11 can be determined from the detected peaks 10. The heart rate 11 is the inverse of the distance between two successive peaks 10. If, for example, the distance between two successive peaks 10 is 1.5 seconds, the heart rate is 1/1.5 Hz=60/1.5 bpm.

Alternatively or additionally, a variability of the heart rate (heart rate variability, HRV) can be acquired, thus the variation of time t between two successive heartbeats 10.

The algorithm can thus be configured to determine cardiac features 6, such as the heartbeat 10 and/or the heart rate 11 and/or the heartbeat interval (beat-to-beat-interval, BBI) and/or the variability of the heart rate and/or the pulse amplitude and/or other cardiac features 6.

These cardiac features 6 can be statistically evaluated in the extraction unit 30 and/or in the classification unit 40. For example, the maxima, minima, mean values, medians, standard deviations, variances, spans, distributions, sums, or differences of the one or the multiple cardiac features 6 can be determined. The extracted cardiac features 6 and/or their statistical evaluations can represent a foundation for determining at least one sleeping phase 110 and/or one waking phase 111.

An inference about the quality of the PPG recording can also be drawn on the basis of the extracted cardiac features 6 and/or their statistical evaluation. If cardiac features 6 such as heart rate 11 or pulse amplitude are outside the physiological range, a poor quality is assigned to the PPG. An inference about the quality of the PPG signal 5 can also be derived from the absolute level of the PPG signal 5. A rapid and/or abrupt change of the absolute level of the PPG signal 5 indicates a poor quality of the PPG. The quality of the cardiac signals 5 can thus be produced not only in the monitoring unit 20, but also downstream in the extraction unit 30 and/or the classification unit 40.

To eliminate disturbances and/or singular events, the extraction unit 30 or an algorithm stored in the extraction unit 30 can carry out artifact detection and/or artifact elimination.

An increase of the measurement sensitivity can moreover be achieved in that the signal evaluation is carried out within at least one predeterminable frequency band. For example, the PPG signals 5 can be bandpass filtered, so that only signals 5 of a predefined frequency band are evaluated. The features in the frequency range, including the spectral power density, which indicates rapid or small changes of the HRV values, are also taken into consideration.

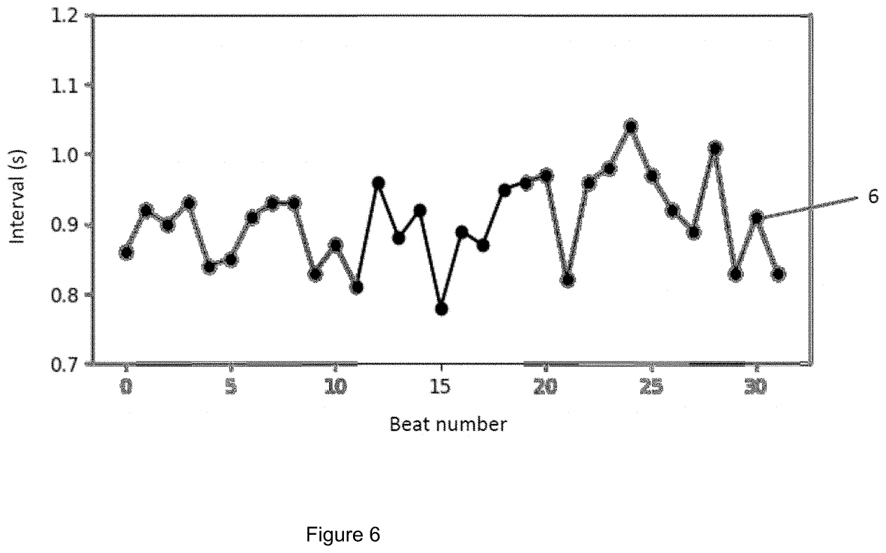

FIG. 6 shows an exemplary representation of an extracted cardiac feature 6 in a coordinate system, wherein the heartbeats 10 are plotted on the X axis and the heartbeat interval in seconds is plotted on the Y axis. The heartbeat interval and/or the variability of the heartbeat interval can be determined as a cardiac feature 6 and can offer, for example, inferences about sleeping phases 110 and/or waking phases 111.

For example, inferences about the vegetative regulation state can be drawn on the basis of such a cardiac intervalogram/rhythmogram shown in FIG. 6. A dominant sinusoidal respiration arrhythmia indicates, for example, that the patient is relatively calm, which can correspond, for example, to a sleeping phase 110.

As already stated herein above, the system 100 can be configured and designed to assign a sleeping phase 110 and/or a waking phase 111 to at least one time period 113 on the basis of the movement signals 8 and/or the cardiac signals 5 and/or the respiration signals 2.

In one preferred exemplary embodiment, the system 100 can also be configured and designed to assign a sleeping phase 110 and/or a waking phase 111 to at least one, preferably each analyzed time period 113 on the basis of the movement features 9 and/or the cardiac features 6 and/or the respiration features 3.

The system 100 preferably assigns a sleeping phase 110 and/or a waking phase 111 to each time period 113 of the time span 112. Due to the selection from the categories physical movement, cardiac activity, and respiration activity, sufficient signals 2, 5, 8 or features 3, 6, 9 are always available according to the invention to preferably assign a sleeping phase 110 and/or a waking phase 111 to each time period 113.

In a simple embodiment, the selected movement signals 8 and/or cardiac signals 5 and/or respiration signals 2 can be passed on directly to the classification unit 40 (see FIG. 3). In one preferred embodiment, the signals 8, 5, 2 can initially be processed by the extraction unit 30 in such a way that the classification unit 40 receives extracted features 3, 6, 9 and/or their statistical evaluations (see FIG. 4).

The classification unit 40 can comprise multiple classification subunits. For example, the classification unit 40 comprises three classification subunits 40i, 40ii, 40iii. The control unit 101 can be configured to activate one of the three classification subunits 40i, 40ii, 40iii in each case based on the results of the monitoring unit 20 in dependence on the presence and/or the quality of the signals 2, 5, 9.

The first classification subunit 40i can be activated if all signals 2, 5, 9 are present in satisfactory quality. The second classification subunit 40ii can be activated if only the signals 5 and 9 are present in satisfactory quality. The third classification subunit 40iii can be activated if only the signals 9 are present in satisfactory quality.

The first classification subunit 40i can then be configured to receive features of all three different categories. The first classification subunit 40i can thus be configured to receive movement features 9 and cardiac features 6 and respiration features 3 and to determine sleeping phases 110 and/or waking phases 111 from the movement features 9 and the cardiac features 6 and the respiration features 3.

The second classification subunit 40ii can be configured to receive features of two different categories. The second classification subunit 40ii can thus be configured to receive movement features 9 and cardiac features 6 and to determine sleeping phases 110 and/or waking phases 111 from the movement features 9 and the cardiac features 6.

The third classification subunit 40iii can be configured to receive features of a single category. The feature which can be available alone is generally from the category of physical movement. The third classification subunit 40iii can therefore be configured in particular to receive movement features 9 and to determine sleeping phases 110 and/or waking phases 111 from the movement features 9.

In alternative exemplary embodiments (not shown here), the system 100 can also comprise further classification subunits, which can utilize the remaining combinations of features, namely, for example, a combination of movement features 9 and respiration features 3 or a combination of cardiac features 6 and respiration features 3 or solely the respiration features 3 or solely the cardiac features 6 to determine sleeping phases 110 and/or waking phases 111 therefrom.

The classification unit 40 or its subunits are accordingly configured to receive signals 2, 5, 8 from the monitoring unit 20 and/or features 3, 6, 9 from the extraction units 30. The evaluation of the signals 2, 5, 8 and/or the features 3, 6, 9 can then take place in the classification unit 40. For this purpose, at least one algorithm for recognizing sleeping phases 110 or waking phases 111 is stored in the classification unit, which analyzes the signals 2, 5, 8 and/or the features 3, 6, 9.

The evaluation of the extracted signals 2, 5, 8 and/or features 3, 6, 9 can take place via a calculation of statistics on the basis of at least one statistical key FIG. 46. For example, minima, maxima, mean values, medians, standard deviations, variances, spans, distributions, sums, or differences of the signals 2, 5, 8 and/or features 3, 6, 9 can be determined. The features 3, 6, 9 are preferably statistically evaluated.

At least one limiting value 42 can be stored in the classification unit 40, on the basis of which the assignment of the sleeping phases 110 and/or waking phases 111 to the time periods 113 is determined. A sleeping phase 110 can be assigned to a time period 113 if the analyzed values (signals 2, 5, 8 and/or features 3, 6, 9) are above the limiting value 42. Conversely, a waking phase 111 can be assigned to a time period 113 if the analyzed values (signals 2, 5, 8 and/or features 3, 6, 9) are below the limiting value 42 and vice versa. A separate limiting value 42 is preferably assigned to each analyzed value, thus each analyzed signal 2, 5, 9 or feature 3, 6, 9. Multiple limiting values 42 can thus be stored and retrievable in the classification unit 40.

The analysis of the different values, thus the signals 2, 5, 8 and/or features 3, 6, 9, can be weighted differently. At least one weighting value 44 can be stored and retrievable for this purpose in the classification unit 40. In one preferred exemplary embodiment, a respective weighting value 44 can be stored for each individual signal 2, 5, 8 and/or feature 3, 6, 9.

The goal is to assign a label “sleeping phase 110” or “waking phase 111” to each time period 113 of the time span 112. The system 100 according to the invention is configured to execute an algorithm for recognizing sleeping phases 110 and waking phases 111 based on the analysis of the respiration features 3 and/or cardiac features 6 and/or movement features 9 and further data/values, for example in the form of weighting values 44 and limiting values 42.

The recognition of a “sleeping phase 110” or a “waking phase 111” is executed, for example, by a machine learning algorithm. For this purpose, initially empirically determined data of living beings are manually evaluated with respect to the sleeping phases 110 or waking phases 111 and made available to the machine learning algorithm.

Certain empirically determined data (the evaluation of features or feature combinations) are thus assigned a respective label “sleeping phase 110” or “waking phase 111” beforehand in the machine learning phase. The machine learning algorithm can then be applied to newly determined, unknown data.

For example, the machine learning algorithm is stored and executed after training by the manually evaluated data in the system 100, for example in the classification unit 40.

The algorithm can compare the different combinations of various respiration features 3, cardiac features 6, and movement features 9 and their respective weighting values 44 to a limiting value 42. Therefore, the sum of (feature A*weighting value A+feature B*weighting value B+ . . . ) can be compared to a respective limiting value 42. The letters A, B . . . each stand in this case for a specific determined respiration feature 3 or cardiac feature 6 or movement feature 9.

The weighting values 44 can be determined beforehand in a machine learning phase by the machine learning algorithm and stored. The machine learning tools are used as a “black box”, into which data (features and labels) and empirically determined parameters can be input. The “black box” thus learns or determines the weighting values 44 for each individual feature 3, 6, 9. The determined weighting values 44 and limiting values 42 can then be used in the algorithm.

The calculation as to whether a sleeping phase 110 or a waking phase 111 exists in a time period 113 can take place as a linear model and/or as a decision tree. In a linear model, in the time span 112 (thus during the running time), for each time period 113, the at least one feature is computed and offset with the previously supplied weighting values 44. On the basis of the above-mentioned linear formula “feature A*weighting value A+feature B*weighting value B+ . . . ”, the result is compared to a previously defined limiting value 42. A decision can be made in this way as to whether a sleeping phase 110 or a waking phase 111 exists.

In the case of a decision tree, a binary tree defined beforehand, having at least one node point, can be used. The respective limiting values 42 are stored at the node points, on the basis of which the decision can be made whether a sleeping phase 110 or a waking phase 111 exists. If the feature X is less than the associated limiting value X, a first path is taken and if the feature X is greater than the associated limiting value X, a second path is taken. Each path has at the end the label “sleeping phase 110” or “waking phase 111”. The features can be calculated for each new time period 113 during the run time. We then traverse the tree and land at a label “sleeping phase 110” or a label “waking phase 111”.

The units 20, 30, 40 are therefore configured and designed as computing units 20, 30, 40, in which algorithms having stored threshold values 22 and/or limiting values 42 and/or weighting values 44 are stored. These threshold values 22 and/or limiting values 42 and/or weighting values 44 are determined on the basis of preceding time periods 113 or time spans 112 in a design time of the system 100.

It is conceivable that the machine learning algorithm of the system 100 achieves learning progress and transmits it via the interface 65 to a server or a cloud. The learning progress data can thus be collected and used to improve or optimize or update the algorithm. This takes place outside the runtime, thus not during a time period 112. It can thus also be configured that the system 100 is updated using data from the server and/or further learning data are made available from time to time. The machine learning algorithm can continue to learn continuously or periodically, for example by way of further manually evaluated data.

Furthermore, FIG. 3 and FIG. 4 show exemplary sequences of the method according to the invention for determining at least one sleeping phase 110 and/or one waking phase 111 for at least one time span 112. In a first step, according to the invention, at least one signal of at least one movement parameter 8 and/or at least one signal of at least one cardiac parameter 5 and/or at least one signal of at least one respiration parameter 2 is acquired. In a further step, the presence of the acquired movement signals 8 and cardiac signals 5 and respiration signals 2 and/or an assessment of the quality of the acquired movement signals 8 and cardiac signals 5 and respiration signals 2 is determined. In a further step, at least one sleeping phase 110 and/or one waking phase 111 is determined from the movement signals 8 and/or the cardiac signals 5 and/or the respiration signals 2.

For this purpose, at least one movement feature 9 can be determined from the movement signals 8 and/or at least one cardiac feature 6 from the cardiac signals 5 and/or at least one respiration feature 3 from the respiration signals 2. At least one sleeping phase 110 and/or one waking phase 111 can then be determined from the movement features 9 and/or the cardiac features 6 and/or the respiration features 3.

It is also considered that the control unit 101 is configured and designed to execute the following method steps to determine at least one sleeping phase 110 and/or one waking phase 111 for at least one time span 112: acquiring at least one signal of at least one movement parameter 8 and/or acquiring at least one signal of at least one cardiac parameter 5 and/or acquiring at least one signal of at least one respiration parameter 2, and determining the presence and/or assessing the quality of the acquired movement signals 8 and cardiac signals 5 and respiration signals 2, and determining at least one sleeping phase 110 and/or one waking phase 111 from the movement signals 8 and/or the cardiac signals 5 and/or the respiration signals 2 on the basis of the determinations and assessments of the monitoring unit 20.

Although the present invention was described in detail on the basis of the exemplary embodiments, it is obvious to a person skilled in the art that the invention is not restricted to these exemplary embodiments. Rather, modifications are possible in such a manner that individual features can be omitted or other combinations of the described individual features can be implemented if the scope of protection of the appended claims is not left. The present disclosure also includes all combinations of the presented individual features.

### LIST OF REFERENCE SIGNS

1 sensor (for example flow sensor)
2 respiration signal
3 respiration feature
4 sensor (for example PPG sensor)
5 cardiac signal
6 cardiac feature
7 sensor (for example actigraphy sensor)
8 movement signal
8X movement signal X axis
8Y movement signal Y axis
8Z movement signal Z axis
9 movement feature
10 heartbeat (peak)
11 heart rate (beat-to-beat interval)
20 monitoring unit
22 threshold value
30 extraction unit
30i first extraction subunit
30ii second extraction subunit
30iii third extraction subunit
40 classification unit
40i first classification subunit
40ii second classification subunit
40iii third classification subunit
42 limiting value
44 weighting value
46 statistical key FIG.
60 memory unit
62 energy supply
64 data transmission unit
65 interface
66 display unit
80 device
82 device belt
84 wired connection
85 fitting
86 hose connection
87 fitting
90 ventilator
92 patient interface
94 hose system
100 system
101 control unit
110 sleeping phase
111 waking phase
112 time span
113 time period
120 sleep quality
t time

What is claimed is:

1. A system having a control unit for determining at least one sleeping phase and/or at least one waking phase for at least one time span, wherein the system comprises
at least one first sensor configured for acquiring at least one movement signal,
at least one second sensor configured for acquiring at least one cardiac signal,
at least one third sensor configured for acquiring at least one respiration signal,
at least one monitoring unit, which is configured to determine and/or assess a presence and/or quality of the acquired at least one movement signal and/or the acquired at least one cardiac signal and/or the acquired at least one respiration signal,
one or more classification units, which are configured to determine at least a sleeping phase and/or a waking phase on the basis of a determination and/or assessment of the at least one monitoring unit from the at least one movement signal and/or the at least one cardiac signal and/or the at least one respiration signal.

2. The system of claim 1, wherein the monitoring unit is configured to receive the at least one movement signal and/or the at least one cardiac signal and/or the at least one respiration signals from the first, second and third sensors, wherein at least one threshold value is stored in the at least one monitoring unit, and wherein the quality of present movement signals and/or cardiac signals and/or respiration signals is assessed on the basis of the at least one predefined threshold value.

3. The system of claim 2, wherein the at least one monitoring unit is configured to select and/or pass on present movement signals and/or cardiac signals and/or respiration signals, which are above or below the respective at least one predefined threshold value.

4. The system of claim 1, wherein the system further comprises one or more extraction units, which are configured to receive selected movement, cardiac and/or respiration signals from the at least one monitoring unit and to determine and/or pass on from selected movement signals at least one movement feature and/or from selected cardiac signals at least one cardiac feature and/or from selected respiration signals at least one respiration feature.

5. The system of claim 4, wherein the at least one determined movement feature is selected from activity count, zero crossing count, median, standard deviation, acceleration minima, acceleration maxima.

6. The system of claim 4, wherein the at least one determined cardiac feature is selected from heart rate, heartbeat, heartbeat interval, heart rate variability, pulse rate, pulse wave, pulse wave amplitude, spectral power density, oxygen saturation (SpO₂ or SaO₂), plethysmograph, increase angle of pulse waves, decrease angle of pulse waves, ratios of increase to decrease angles of pulse waves, durations of pulse wave increases, durations of pulse wave decreases, ratios of increase to decrease durations, pulse wave maxima, pulse wave minima, PTT (pulse transit time), CWF (continuous wave fluctuation), pulse amplitude.

7. The system of claim 4, wherein the at least one determined respiration feature is selected from respiration frequency, peak flow, variability, respiratory gas volume per breath, respiratory gas flow, contour of the respiratory gas flow, inspirational or expirational tidal volume, restoration, inspiration duration, expiration duration, leakage, ratio of inspiration to expiration duration, respiratory minute volume.

8. The system of claim 4, wherein the one or more classification units are configured to receive signals movement, cardiac and/or respiration signals from the at least one monitoring unit and/or movement, cardiac and/or respiration features from the one or more extraction units, wherein the one or more classification units are configured to statistically evaluate the movement, cardiac and/or respiration signals and/or the movement, cardiac and/or respiration features on the basis of at least one statistical key figure.

9. The system of claim 8, wherein the statistical key figure is selected from minimum, maximum, mean value, median, standard deviation, variance, span, distribution, sum, difference.

10. The system of claim 1, wherein the one or more classification units are configured to determine at least one sleeping phase and/or at least one waking phase from the movement, cardiac and/or respiration signals and/or from statistical evaluations thereof.

11. The system of claim 4, wherein the one or more classification units are configured to determine at least one sleeping phase and/or at least one waking phase from the movement, cardiac and/or respiration features and/or statistical evaluations thereof.

12. The system of claim 1, wherein the one or more classification units comprise three classification subunits, wherein the at least one control unit is configured to activate one of the three classification subunits in each case based on results of the at least one monitoring unit in dependence on the presence and/or the quality of the movement, cardiac and/or respiration, wherein a first classification subunit is activated if all of movement, cardiac and respiration signals are present in satisfactory quality, wherein a second classification subunit is activated if only movement and cardiac signals are present in satisfactory quality, and wherein a third classification subunit is activated if only movement signals are present in satisfactory quality.

13. The system of claim 12, wherein the first classification subunit is configured to receive movement features and cardiac features and respiration features and to determine sleeping phases and/or waking phases from the movement features and the cardiac features and the respiration features and/or wherein the second classification subunit is configured to receive movement features and cardiac features and to determine sleeping phases and/or waking phases from the movement features and the cardiac features, and/or wherein the third classification subunit is configured to receive movement features and to determine sleeping phases and/or waking phases from the movement features.

14. The system of claim 1, wherein the time span is predeterminable, and/or wherein the time span is at least 10 minutes and/or wherein the time span corresponds to a duration of a night's sleep and/or wherein the time span is divided into one or more time periods which are at least 10 seconds long.

15. The system of claim 4, wherein the system is configured and designed to assign a sleeping phase and/or a waking phase to at least one time period on the basis of the at least one movement signal and/or the at least one cardiac signal and/or the at least one respiration signal and/or wherein the system is configured and designed to assign a sleeping phase and/or a waking phase to at least one time period on the basis of the at least one movement feature and/or the at least one cardiac feature and/or the at least one respiration feature.

16. The system of claim 1, wherein at least one limiting value is stored in the one or more classification units, and wherein an assignment of a sleeping phase and/or a waking phase to a time period is determined on the basis of the at least one limiting value.

17. The system of claim 16, wherein a sleeping phase is determined for a time period if the values are above one or multiple limiting values and wherein a waking phase is determined for a time period if the values are below one or the multiple limiting values, or vice versa, and/or wherein an assignment of sleeping phases and/or waking phases to time periods is determined on the basis of multiple limiting values, respective limiting values being stored and retrievable in the one or more classification units for each movement, cardiac and/or respiration feature, and/or wherein the one or more classification units are configured to determine sleeping phases and/or waking phases for each time period from at least one movement, cardiac and/or respiration.

18. The system of claim 4, wherein an analysis of different movement, cardiac and/or respiration features is weighted differently, for which purpose at least one weighting value is stored and retrievable in the one or more classification units.

19. The system of claim 1, wherein the system is configured to determine a sleep quality based on a number of sleeping phases and/or the number of waking phases.

20. A method for determining at least one sleeping phase and/or at least one waking phase for at least one time span, wherein the method comprises
acquiring at least one signal of at least one movement parameter and/or
acquiring at least one signal of at least one cardiac parameter and/or
acquiring at least one signal of at least one respiration parameter, and
determining a presence and/or assessing the quality of acquired movement signals and acquired cardiac signals and acquired respiration signals, and
determining at least one sleeping phase and/or at least one waking phase from the acquired movement, cardiac and respiration signals on the basis of determinations and assessments of a monitoring unit.
